(19) United States
(12) Patent Application Publication
(10) Pub. No.: US 2008/0317780 A1
(43) Pub. Date: Dec. 25, 2008

(54) VIRAL VACCINE AND METHOD FOR PREPARATION THEREOF

(75) Inventors: Henry FLISS, Ottawa (CA); Anil DUDANI, Kanata (CA)

Correspondence Address: BORDEN LADNER GERVAIS LLP;Anne Kinsman, WORLD EXCHANGE PLAZA, 100 QUEEN STREET SUITE 1100, OTTAWA, ON, K1P 1J9, CA

(73) Assignee: NATIONAL RESEARCH COUNCIL OF CANADA, Ottawa (CA)

(21) Appl. No.: 11/925,340

(22) Filed: Oct. 26, 2007

Related U.S. Application Data
(60) Provisional application No. 60/863,180, filed on Oct. 27, 2006.

(51) Int. Cl. A61K 39/00; C12N 5/06; A61P 31/00

(52) U.S. Cl. 424/208.1; 424/204.1; 424/207.1; 424/235.1

(57) ABSTRACT

The invention relates to a viral vaccine, especially a retroviral vaccine, and methods of forming such a vaccine. The vaccine is formed from a whole-killed virus suspension in a cell lysate or culture medium, wherein the virus is killed by exposure to a chloramine compound at a level adequate to inactivate zinc finger proteins. The chloramine compound may be taurine chloramine, adenosine chloramine, phenylalanine chloramine, and alanine chloramine. The vaccine may be used as a prophylactic or therapeutic vaccine and may be developed from a subject's own strains of virus so as to be considered an autologous vaccine.

This application claims priority from U.S. Ser. No. 60/683,180 filed on Oct. 27, 2006, which is incorporated herein by reference in its entirety.

### FIELD OF THE INVENTION

The present invention relates generally to a killed whole-virus vaccine for use against viruses, especially retroviruses, and to a method for preparation of the vaccine.

### BACKGROUND OF THE INVENTION

A key problem in the battle to prevent HIV infection in humans is the development of an effective vaccine. Killed whole-virus vaccines have been weapons of choice in attempts to immunize patients against viral infections. An advantage of this approach is that all of the viral antigens are presented in a completely “native” conformation and composition. However, killed whole-virus vaccines are notoriously hard to prepare. The key problem in the preparation of such vaccines is that the methods used for inactivating the virus invariably cause changes in the conformation or composition of the virus. For example, inactivation procedures involving such means as heat or formalin adversely affect the viral envelope proteins. The use of such inactivated vaccines therefore raises an immune reaction that does not necessarily recognize the native virus and is therefore ineffective. Thus, a disadvantage of the killed whole-virus vaccine approach is the difficulty in maintaining the “native” configuration of the virus during the chemical inactivation required to render the product safe for humans. Intense efforts are being made to find agents that are capable of completely inactivating whole viruses, but are at the same time mild enough not to appreciably disturb the native conformation of the viral envelop.

International Patent Application No. PCT/CA01/01421 (Fliss) “Chloramines as antiviral agents”, published Apr. 11, 2002, teaches the use of chloramines as antiviral agents useful in sterilizing blood. The manner in which zinc finger proteins of a retrovirus may be inactivated by chloramines is discussed in this publication. Briefly, the oxidative effect of chloramines allows selective attack and destruction of conserved portions of a retrovirus, specifically the cysteine-rich zinc-finger regions. The conservation of susceptible zinc finger motifs is a property of all retroviruses. The oxidative attack results in removal of zinc from cysteine-zinc thiolate bonds in the zinc-finger regions, thereby preventing viral infectivity. This publication makes no mention, however, of vaccine preparation.

Nagl, Gottardi and co-workers have illustrated the use of a specific chloramine, taurine chloramine (N-chlorotaurine, chlorotaurine) as an antimicrobial agent to counter such conditions as conjunctivitis, herpes simplex and adenoviruses in their publications: Nagl, M., Teuchner, B., Pottinger, E., Ulmer, H., and Gottardi, W. “Tolerance of N-chlorotaurine, a new antimicrobial agent, in infectious conjunctivitis—A phase II pilot study”. Opthalmologica 2000, 214:111-114. Nagl, M., Hess, M. W., Pfaller, K., Hengster, P., and Gottardi, W. “Bactericidal activity of micromolar-chlorotaurine: Evidence for its antimicrobial function in the human defense system” Antimicrob. Agents Chemother. 2000, 44:2507-2513; and Teuchner et al. “Tolerance and efficacy of the new antimicrobial agent N-chlorotaurine in viral keratoconjunctivitis” Annual Meeting of the Association for Research in Vision and Opthalmology, (abstract) Mar. 15, 2001 page S578 (XP001027296).

Further, other publications suggest therapeutic uses of taurine chloramine, for example German Patent applications DE 197 12 565 A (Steif) and DE 198 16 102 C (Gottardi). However, none of these publications suggests utility of chloramines in vaccine preparation.

Chemical inactivation of a retrovirus by targeting zinc finger proteins is discussed by Arthur et al. (Aids Res. Human Retrovir 1998, 14:S311-S319) and by Rossio et al. (J. Virol. 1998, 72:7992-8001). However, the compound used for inactivation is 2,2′-dithiodipyridine. The compound 2,2′-dithiodipyridine (also known as Aldrithiol-2) is a thiol-reactive agent that targets zinc fingers in nucleocapsid proteins, and would, in itself, be toxic to cells or a patient if administered on its own. Thus, following treatment with 2,2′-dithiodipyridine, it is necessary to isolate the virus from the residual 2,2′-dithiodipyridine, or other similar toxic agents, in order to render a preparation safe for vaccine use.

Chemical inactivation of a retrovirus by targeting zinc finger proteins is also discussed by Turpin et al. in U.S. Pat. No. 6,706,729. However, the compounds used for inactivation are thiolester derivatives. It is proposed that thiolester-inactivated retrovirus may be used for the preparation of vaccines. However, it is required that the virus be first isolated and then treated with the thiolester, introducing a viral purification step that inevitably causes changes in viral conformation. In the publication of Robbins, G. K., et al. “Augmentation of HIV-1-specific T helper cell responses in chronic HIV-1 infection by therapeutic immunization” AIDS 1993, 17:1121-1126, an inactivated HIV-1 vaccine was used in human subjects. HIV positive subjects were treated with an inactivated, envelope-depleted (gp120-depleted), HIV-1 virus coupled with incomplete Freund's adjuvant. The depleted virus was inactivated using a combination of chemicals and radiation. This had the effect of increasing immune response in these subjects. However, since the immunogen was envelope-depleted, it is not comparable to a whole-killed virus.

The publication of Matteucci, D. et al. “Vaccination protects against in vivo-grown feline immunodeficiency virus (FIV) even in the absence of detectable neutralizing antibodies” J. Virol. 1996, 70:617-622 describes a FIV grown in lymphoid cells, which cells are then fixed in 1.25% formaldehyde for 24 h at 37° C. prior to purification of the inactivated FIV and injection into cats. Although this approach showed protection against subsequent infection, formaldehyde-fixed cells are unlikely to be acceptable for use in the preparation of human vaccines.

Henderson et al. discusses a method of identifying compounds that inactivate HIV-1 and other retroviruses by attacking zinc fingers (specifically CCHC zinc fingers) in U.S. Pat. No. 6,001,555. This document discusses utility of such compounds, once identified, for delivery to a patient infected by a virus, or as a preventative topical lubricant that would prevent transmission of such a retrovirus.

Vaccines are generally defined as suspensions of dead, attenuated, or otherwise modified microorganisms (viruses, bacteria, or rickettsiae) for inoculation to produce immunity to a disease by stimulating the production of antibodies. A variety of different vaccine types exist which may be pure or comprise a combination of elements, such as for example, vaccines containing viral peptides, DNA (or “naked” DNA), live vectors such as a bacteria carrying a non-native gene of interest, live-attenuated vaccines, or whole-killed (inactivated) vaccines. It is recognized that a whole-killed viral vaccine is an optimal approach because the viral antigens are presented in “native” form. However, there are safety concerns and problems relating to the use of a whole-killed virus, such as the reactivation of the virus, the harsh chemical treatment and/or purification required to remove the killing agent from the cell lysate suspension, and the difficulty encountered in maintaining the virus particles in a “native” form that would thus allow recognition of the live virus, to name a few. There is a need for a viral vaccine, especially a retroviral vaccine, that overcomes these problems commonly experiences with whole-killed viral vaccines.

### SUMMARY OF THE INVENTION

It is an object of the present invention to obviate or mitigate at least one disadvantage of previous viral vaccines, and in particular whole-killed viral vaccines.

According to an embodiment of the invention, there is provided a viral vaccine, especially a retroviral vaccine, comprising a cell lysate or culture medium containing a whole-killed virus suspension and a pharmaceutically acceptable carrier, said virus having been killed by exposure to a chloramine compound at a level adequate to inactivate zinc finger proteins.

The concentration of chloramine compound employed depends upon the precise nature of the compound and the nature of the virus. Typically the range is within 1 to 100 mM, more especially 2 to 50 mM, particularly 2 to 10 mM, preferably about 2 to about 5 mM.

The time of exposure of the virus to the chloramine compound is typically within the range of 1 minute to about 6 hours, especially 2 minutes to about an hour, particularly 5 to 45 minutes, especially about 30 minutes.

A further embodiment of the invention provides a method for forming a viral vaccine comprising the steps of: obtaining a sample of a virus; and exposing said virus sample to a chloramine compound in a quantity adequate to inactivate zinc finger proteins of said virus. Viral vaccines formed according to this embodiment of the invention are encompassed by the invention.

Additionally, an embodiment of the invention provides a method of autologous vaccination of a subject against a virus, said method comprising the steps of: obtaining a sample of a virus from a subject; exposing said virus sample to a chloramine compound in a quantity adequate to inactivate zinc finger proteins of said virus to form a vaccine; and providing said vaccine to the subject.

The present invention provides a vaccine for use against viruses in which essential zinc finger structures are highly conserved. Chloramines such as taurine chloramine, adenosine chloramine, alanine chloramine, phenylalanine chloramine, and others may be used. The vaccine may be prepared from a whole-killed virus, specifically, the target virus, especially the target retrovirus. The vaccine may consist of the whole-killed virus and the original cell lysate or culture medium in which it was killed. Thus, the entire chloramines-treated cell lysate from virus-infected cells may be used as prepared. Alternatively, the vaccine may consist of whole-killed virus that had been purified from the chloramines-treated cell lysate. The vaccine may be prepared as an autologous vaccine wherein a subject provides a blood sample from which the virus is obtained, the whole-killed viral vaccine is prepared as a cell lysate by exposure to a chloramine, and the preparation so developed is administered back to the subject. This embodiment has the advantage that the vaccine will be specific to the viral strains currently infecting the subject.

An advantage of certain embodiments of the invention is its use of one or more mild oxidants (one or more of the chloramines) to inactivate viral suspensions, thereby avoiding any direct alterations to the viral native conformation that would normally attend treatment with harsher inactivating agents. In addition, the use of non-toxic chloramines precludes the need to isolate and purify the killed virus from the residual inactivating agent prior to the incorporation of the virus into a vaccine preparation. Such purification steps would inevitably result in alterations to the native viral conformation. Consequently, the use of one or more mild chloramines permits the treatment of virus suspensions directly in infected cell lysates or culture medium, and permits the direct incorporation of the treated suspensions in vaccine preparations without additional viral purification steps.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the figures.

FIG. 1 illustrates spleen weight data provided in Table 1, PART A.

FIG. 2 illustrates spleen weight data of unvaccinated versus vaccinated animals provided in Table 1, PART B.

FIG. 3 illustrates spleen weight data of unvaccinated versus vaccinated animals in both prophylactic and therapeutic models

FIG. 4 illustrates the activity of the chloramine of ammonia (monochloramine) “A”; the oxidant hypochlorous acid (HOCl) “B”; taurine chloramine “C”; and the chloramine of the amino acid alanine “D”, on zinc-finger disruption.

FIG. 5 illustrates the activity of “A” the amino acid phenylalanine when compared to “B” phenylalanine chloramine, and “C” the oxidant hypochlorous acid (HOCl), on mobilization of zinc in the zinc-finger disruption assay.

FIG. 6 illustrates the activity of “A” the chloramine of adenosine, versus “B” hypochlorous acid (HOCl) on mobilization of zinc in the zinc-finger disruption assay.

FIG. 7 illustrates the ability of taurine chloramine to inactivate HIV in human blood at 2 mM (upper panel) and 5 mM (lower panel).

### DETAILED DESCRIPTION

The use of chloramine-inactivated retrovirus preparations as whole-killed viral vaccines is described herein. Taurine chloramine is a natural compound (produced by neutrophils), and is a mild oxidant. Treatment with taurine chloramine, as well as other chloramines, results in complete inactivation of viruses that contain essential zinc fingers, such as retroviruses, while allowing the killed virus to retain its native conformation and, therefore, its ability to stimulate an immune reaction against the live infectious virus. Further, the use of a viral suspension in cell lysate or culture medium, rather than purified virus, to prepare a vaccine is described herein. Preparation of simple and effective vaccines against viral infections, such as HIV, can be achieved according to the invention.

According to the invention, it has been found that chloramine agents, such as taurine chloramine, are capable of completely inactivating whole viruses, but are mild enough not to appreciably disturb the native conformation of the viral envelop.

According to an embodiment of the invention, there is provided a viral vaccine, such as a retroviral vaccine, comprising a cell lysate or culture medium containing a whole-killed virus suspension and a pharmaceutically acceptable carrier, the virus having been killed by exposure to a chloramine compound at a level adequate to inactivate zinc finger proteins. The viral vaccine may additionally comprise a chloramine compound such as taurine chloramine, adenosine chloramine, phenylalanine chloramine, and alanine chloramine. The vaccine may be a prophylactic vaccine or a therapeutic vaccine. It is also within the scope of the invention that the vaccine may be an autologous vaccine.

A further embodiment of the invention provides a method for forming a viral vaccine comprising the steps of: obtaining a sample of a virus; and exposing the virus sample to a chloramine compound in a quantity adequate to inactivate zinc finger proteins of the virus. The virus may be a retrovirus such as HIV. The viral sample may be exposed to a chloramine at any concentration capable of viral killing, for example from about 1 to 10 mmol/L. After the step of exposing the virus sample to a chloramine compound, a whole-killed virus may be obtained, and this may be used as a vaccine. Alternatively, extracts may be used as a vaccine.

According to a further embodiment of the invention, there is provided a method of autologous vaccination of a subject against a virus, having the following steps: obtaining a sample of a virus from a subject; exposing the virus sample to a chloramine compound in a quantity adequate to inactivate zinc finger proteins of the virus to form a vaccine; and providing the vaccine to the subject. The step of obtaining a sample of a virus may involve amplifying the virus in cell culture and preparing extracts of the cell culture to be used in the step of exposing the virus sample to a chloramine compound. Such cell culture may be autologous or heterologous cell culture.

Taurine chloramine can effectively kill high titers of HIV in human blood (greater than 6 logs of inactivation). Yet taurine chloramine is a mild oxidant that does not appear to alter the viral envelope conformation or composition. Taurine chloramine, as well as other chloramines, can therefore effectively kill high titers of a retrovirus, thereby rendering it safe for clinical use, and at the same time retaining the native conformation of the virus and therefore its ability to stimulate an immune reaction against the live infectious virus. A property that makes chloramines, such as taurine chloramine, appropriate inactivating agents is the selectivity of chloramines for the cysteine residues in proteins. All retroviruses, such as HIV, contain essential cysteine residues in their proteins. For example, one retroviral nucleocapsid protein that is essential for viral infectivity (NCp7) contains a highly conserved zinc finger structure that consists of essential cysteine residues. Taurine chloramine can therefore target and selectively oxidize these essential cysteines, thereby eliminating the infectivity of the retrovirus, yet not disturbing the remainder of the protein. In addition, since retroviral envelope proteins do not contain cysteine residues capable of reacting with chloramines (Arthur et al. (Aids Res. Human Retrovir 1998, 14:S311-S319) and Rossio et al. (J. Virol. 1998, 72:7992-8001)), chloramines do not alter the native conformation of the highly immunogenic envelope proteins.

LP-BM5 viral preparations treated with taurine chloramine were shown to be completely inactive using in vitro culture techniques, yet these preparations remained strongly immunogenic as demonstrated by the production of a very strong immunity to the live virus in mice.

According to the invention, the vaccine is prepared from a viral suspension in cell lysate or culture medium, rather than from isolated and purified virus. A difficulty in preparing a normal vaccine is the need to isolate and purify the virus prior to its inactivation, as purification procedures typically result in the loss of key envelope proteins, thus rendering the final vaccine ineffective. However, according to the invention, a viral suspension in cell lysate or culture medium can be used to accomplish effective immunization. Although there are some safety issues to consider prior to the clinical use of cell lysates, these issues can be addressed by one of skill in the art, and do not diminish the benefits of this approach.

The whole-killed viral vaccine approach taken according to the invention overcomes many of the concerns and safety issues surrounding previous whole-killed viral vaccines. According the invention, the risk of re-activation is traversed, because chloramines, such as taurine chloramine are effective killers of viruses such as HIV. Harsh chemical treatment & purification is avoided because chloramines such as taurine chloramine are mild, and naturally occurring. This compound targets thiols only, and thus leaves the remainder of the virus intact. The use of viral suspensions in cell lysates or culture medium negates further purification, which in itself may be harmful to the conformation of the killed virus. This overcomes the previous problem that whole-killed viruses are difficult to maintain in their “native” form. Taurine chloramine does not appear to alter surface protein integrity when used according to the invention, unlike prior art methodologies.

MAIDS is a predictive and widely accepted model for studying HIV. MAIDS responds to anti-HIV drugs. One skilled in the art would understand that the whole-killed virus vaccine approach carries through to subjects having HIV, and several research groups working in this area use MAIDS as a model. Further support of the parallels and adequacy of the model of MAIDS versus HIV can be found in the prior art, for example in International Patent Application No. PCT/CA01/01421 (Fliss) “Chloramines as antiviral agents”. Published Apr. 11, 2002.

The instant invention provides a simple, fast approach to vaccine formation by using a mild, effective inactivating agent (taurine chloramines or other chloramines) that targets essential zinc fingers in viruses.

According to an embodiment of the invention, an autologous vaccine may be prepared and used. An autologous vaccine is a “custom-made” therapeutic vaccine prepared in order to specifically target a patient's own viral strains. A procedure for autologous vaccine preparation could be illustrated using HIV as a specific example. An HIV-infected patient is immunized with a vaccine made of his or her own HIV strains by first collecting blood from the subject. The blood is then used to isolate and culture infected PBMC, the HIV viral strains are amplified in the patient's own PBMC, and a PBMC extract (lysate) is prepared. This extract is inactivated with taurine chloramine (or another chloramine), and subsequently, the subject is immunized with his or her “custom-made” vaccine.

When an autologous vaccine is prepared, the approach according to the invention is extremely safe, as a subject's own blood or tissue samples are used. This has the additional advantage of avoiding mutation problems that may arise from vaccines prepared in advance or from viral samples taken in other populations which may be geographically remote and that may have mutated significantly relative to the viral strains found in any individual subject.

The instant invention also has the advantage of not requiring purification of the virus because it can be inactivated directly in cell lysates or culture medium prior to injection in a subject. Taurine chloramine (and other chloramines) may be used at levels that are not harmful to a subject.

Not only can the invention be used as a therapeutic vaccine to alleviate, or attenuate viral infection in an infected individual, but it may also be used as a prophylactic vaccine to immunize uninfected patients. When immunizing uninfected patients, viral strains may be derived from a particular geographic location, so that the specific strain, or strains, of the virus most likely to be found in infected individuals of that location are the ones upon which the vaccine is based. Further, if a subject can identify the strain(s) of the infected individual carrying the virus to which they were exposed, a vaccine could be produced on the basis of the identified strain(s), or a sample derived from the individual to which the subject was exposed.

The invention is of use as a vaccine against HIV, as well as other retroviruses since most known retroviruses have essential and highly susceptible zinc finger proteins. Other viruses or non-viral pathogens could also be targeted with this invention, provided they have susceptibility to inactivation through attack on zinc finger proteins. One of skill in the art could easily determine susceptibility of a virus or other pathogen to zinc finger attack.

Although one advantage of the present invention is that the killed virus need not be further purified from the cell lysate or culture medium, it is certainly within the scope of the invention to permit purification of the virus, if it is desired. In either case, the resulting vaccine may be used as a prophylactic vaccine or as a therapeutic vaccine which may be autologous, or geographically limited to a region (or “geologous”), so as to limit the mutations the retrovirus has undergone and increase the likelihood of efficacy in an individual in a geographical region.

An adjuvant may be used, which is pharmaceutically acceptable for administration to the subject in question. This may be advantageous in such cases where the adjuvant helps to increase the immunogenicity of the vaccine.

### EXAMPLE 1

### Protective Effect of Immunization with Chloramine-Killed Virus on Mice Infected with MAIDS Virus

We have shown that the murine immunodeficiency (MAIDS) virus, LP-BM5, can be completely inactivated with 5 mM taurine chloramine, a natural product and a mild oxidant, and that the inactivated virus can then be used to immunize uninfected mice against subsequent infection with the live LP-BM5 virus, as well as provide protection to already infected mice. The following are the details of the experiment.

### Methods

Virus inactivation. LP-BM5 retrovirus or murine AIDS (MAIDS) was grown in cultured murine SC-1 cells using standard in vitro tissue culture techniques. The cells were then harvested and lysed, and the viral titer was adjusted to around 5×10⁵ PFU/ml. Aliquots of the cell lysate containing the viral suspension were then inactivated with taurine chloramine. Typically, this inactivation was performed by combining 2 ml of cell lysate with 2 ml of 10 mM taurine chloramine (final concentration 5 mM) for 30 min at 37° C. The inactivated lysate was shown to contain no active virions by plating serial dilutions of the lysate in SC-1 cultures.

Mouse immunization. Mice were inoculated intraperitoneally (i.p.) with a bolus of 200 μl of the taurine chloramine-treated lysate (2.5×10⁴ “PFU” of inactive virus per injection), with or without Ribi adjuvant. Four weeks later, the mice received a second i.p. booster injection of 200 μl of the taurine chloramine-treated lysate, with or without the Ribi adjuvant. Seven days after the booster injection, the mice were challenged i.p. with 100 μl containing 500 PFU of live virus. The mice were killed on week 8 after the live virus challenge, and the spleen was isolated and weighed to determine the extent of infection.

Murine Leukemia Virus (MuLV) LP-BM5. LP-BM5 was obtained from the NIH AIDS Research and Reference Reagent Program in the form of persistently infected SC-1 feral mouse embryonic fibroblast cells (SC-1/MuLV LP-BM5, catalog #1215). LP-BM5 is described by the NIH AIDS Research and Reference Reagent catalog as “a mixture of defective, ecotropic and mink cell focus-forming (MCF) MuLVs. Persistently infected SC-1 feral mouse embryo cells release BM5 def, the replication-defective, disease-inducing component of LP-BM5 MuLV, as well as ecotropic and MCF MuLV (replication-competent B-tropic MuLVs that act as helper virus for BM5 def; the ecotropic component is present at higher titer than the MCF). Cell-free harvests induce splenomegaly and lymphadenopathy within 4-6 weeks in adult C57BL/6 mice inoculated intraperitoneally.”

Propagation and Harvesting of LP-BM5. The infected SC-1 cells were grown at 37° C., 5% CO₂, in propagation medium: McCoy's basal medium supplemented with 1% penicillin-streptomycin, 90% L-glutamine, and 10% heat-inactivated fetal bovine serum. The cultures were split when they reached confluence using standard trypsin/EDTA techniques. Pools of LP-BM5 were prepared by mixing 1×10⁶ infected SC-1 cells with 1×10⁶ uninfected SC-1 cells (ATCC, catalog #CRL-1404) in 20 ml of propagation medium in a 75 cm² flask. The medium was changed on days 1, 3, and 5 with 30 ml of fresh medium.

LP-BM5 was harvested from both the medium and cells on the day following a medium change, when the SC-1 cultures were near confluence. Working at 4° C., the medium was collected, and the infected cells were harvested by scraping into a small volume of medium (4 ml). The harvested cells were broken with one cycle of freeze thawing, and were then further disrupted by 2-3 aspirations into a syringe through a 22-gauge needle. To collect the entire viral fraction, the disrupted cell preparation was combined with the previously collected medium and the entire suspension was subjected to centrifugation at 2500 rpm for 20 min. The viral preparation in the supernatant was aliquoted and was kept frozen at −80° C. Uninfected cell extracts were prepared in identical fashion from uninfected SC-1 cells.

Plaque Assay For LP-BM5. D-56 Murine fibroblasts (1×10⁵, a gift from Dr. Alan Rein, National cancer Institute, Bethesda, Md.) were transferred in 4 ml McCoy's medium to a 6 cm diameter tissue culture dish containing a grid. The cells were mixed with a back and forth motion for one min to achieve even distribution, and were then incubated overnight at 37° C. DEAE-Dextran was then added to the medium to a final concentration of 20 μg/ml. After 20 min, the medium was aspirated off, the cells were washed with 2 ml of McCoy's, and 0.5 ml of virus suspensions were added. The virus suspensions were prepared by serial dilution in McCoy's medium of the viral preparations prepared above: e.g. 1/10, 1/100, etc. The tissue culture dishes were then incubated at 37° C., 5% CO₂, for 30 min. Six ml of McCoy's was then added to each dish which was then incubated for 5 days. The foci (plaques) on each dish were then counted and used to calculate the titer of the virus in PFU/ml. Viral suspensions of 5×10⁵ PFU/ml were prepared from the original viral stocks and aliquots were kept at −80° C.

Inactivation of viral preparations with taurine chloramine. Stock aqueous solutions of the microbicide, taurine chloramine, were prepared at concentrations of 1 M or 100 mM and aliquots were kept frozen at −80° C. For viral inactivation, aliquots of the viral stock (5×10⁵ PFU/ml) and aliquots of the microbicide were thawed immediately prior to inactivation. The microbicide was diluted to 10 mM in water, and equal volumes (e.g. 2 ml) of the viral stock and 10 mM microbicide were combined in a 15 ml polypropylene tube, and were mixed to give final concentrations of 5 mM microbicide and 2.5×10⁵ PFU/ml LP-BM5. The tube was then incubated at 37° C. for 30 min. Extracts from uninfected SC-1 cells were subjected to an identical inactivation procedure. Using the plaque assay described above, the inactivated lysate was shown to contain no active virions.

Infection of mice with LP-BM5. Mice (C57BL/6, female, 4-6 weeks old, 18-22 g, Charles River) were housed at 3-4 per cage and were allowed 5 days of acclimatization in the animal housing facilities prior to enrolment in an experimental procedure. The cages, food, bedding and water were all sterilized by autoclaving before use, and the cages were kept inside a containment area. Infection of mice with the LP-BM5 viral preparations resulted in a Murine Acquired Immunodeficiency Syndrome (MAIDS). Infection was achieved with an i.p. injection of 500 PFU of LP-BM5 in 0.1 ml.

Immunization of mice. Mice were immunized with two injections of taurine chloramine-inactivated LP-BM5 (prepared as described above) either with, or without, the adjuvant Ribi (MPL+TDM Emulsion, R-700, CORIXA, 553 Old Corallis Road, Hamilton, Mont. 59840 USA). Each Ribi Adjuvant System (RAS) vial contained 0.5 mg Monophosphoryl Lipid A (MPL), 0.5 mg Synthetic Trehalose Dicorynomycolate (S-TDCM), 0.040 ml Squalene (hexamethyl-tetracosahexane), and 0.004 ml Monooleate (Tween 80) in a stable oil/water suspension which is reconstituted with 2 ml PBS prior to use. Inactivated LP-BM5 was mixed with an equal volume of reconstituted Ribi adjuvant, and 0.2 ml of this mixture (2.5×10⁴ “PFU” of inactive virus) was injected i.p. per mouse. The first injection was administered on Day 0, and a second, booster injection was administered on Day 30. In mice that did not receive Ribi, the adjuvant was replaced with an equal volume of PBS.

Challenging Immunized mice with LP-BM5. For the prophylactic model, immunized mice (see above) were challenged on Day 38 with active LP-BM5 as described above under “Infection of mice with LP-BM5”. The mice were killed on week 8 and week 16 after the live virus challenge, and the lymph nodes and spleen were harvested and analyzed for extent of infection as described below. For the therapeutic model, mice were first infected with LP-BM5 as described above. The infected mice were then inoculated with the first vaccine bolus on either day 2 or day 7 post-infection, and received the second bolus of vaccine 30 days following the first inoculation. The mice were sacrificed on week 8 following the second bolus administration and were examined for pathological evidence of MAIDS.

Harvesting of tissues from mice. Mice were anesthetized and were killed by exsanguination through cardiac puncture. The blood was drawn into sodium citrate-containing syringes. The lymph nodes were then removed under aseptic conditions, were frozen rapidly in a dry ice-ethanol solution, and were stored at −80° C. The peritoneal cavity was opened under aseptic conditions and the spleen was removed and weighed. Spleen weight was used to determine the severity of viral infection. After weighing, the harvested spleen was cut rapidly into several fragments. Some of the fragments were frozen rapidly as above and were stored at −80° C. for future RNA extraction (see below). The remaining portions of spleen were transferred to 2 ml of McCoy's medium for cytokine studies. The collected blood was centrifuged, the plasma and leukocyte fractions were collected and aliquoted, and were kept frozen at −80° C.

RNA Extraction and RT-PCR. RNA extraction and RT-PCR analysis of the extracted RNA were used to determine qualitatively the presence of MAIDS viral RNA in the tested tissues. RNA Extraction from cells (SC-1 and splenocytes) was performed using the RNeasy™ mini kit (Qiagen™ cat #74104 and QIAshredder™ column cat #79654). Frozen or fresh cells were transferred to a 15 ml polypropylene tube containing 10 ml of medium. The cells were pelleted by centrifugation and were washed with 5 ml of medium. The pellet was then subjected to RNA extraction using the protocol recommended by the manufacturer. RNA was also extracted from fresh or frozen tissues using the RNeasy mini kit using the manufacturer's protocol. RT-PCR was performed on the extracted RNA using standard techniques and the LP-BM5 primers whose sequence was obtained from Hulier E, et al. “Quantitative assessment of murine retrovirus LP-BM5 infection in MAIDS by PCR and anion exchange HPLC” J. Virol. Methods. 1996, 60:109-117. The following primers were used:

forward primer (upstream or sense):

(SEQ ID NO: 1)

CCT TTA TCG ACA CTT CCC TT;
and
reverse primer (downstream):

(SEQ ID NO: 2)

CCG CCT CTT CTT AAC TGG TC.

### Results

Table 1 illustrates spleen weight data in animals either not immunized or immunized with inactivated LP-BM5 virus (MIC-LP-BM5) prepared as described above, either with or without Ribi adjuvant. Animals were then either uninfected or infected with live LP-BM5. Spleen weight was determined at 8 weeks after infection.

Table 1 also illustrates the results of RT-PCR analysis of LP-BM5 for immunized and non-immunized mice infected or uninfected with MAIDS (“PCR Results LP-BM5” column). RNA extraction from spleen was conducted with 20-30 mg of spleen tissue, and from lymph node tissue. Spleen and lymph node tissues gave the same result.

These data show that non-immunized mice infected with the live virus (Control Virus, n=4) had greatly enlarged spleens when compared to uninfected mice (Control, n=3) (378±83 mg vs. 105±3 mg, respectively). Mice that were immunized with the inactivated virus without Ribi adjuvant and were subsequently challenged with the live virus (Ev1, n=5) were not protected from subsequent infection with live virus, as evidenced by enlarged spleens (spleen weight 376±159 mg). However, mice that were immunized with the inactivated virus with the Ribi adjuvant and subsequently challenged with the live virus (Ev2, n=5) were protected, as evidenced by the spleen weight (spleen weight 126±42 mg), which did not differ significantly from the spleen weights of the control uninfected group or the immunized/Ribi uninfected group (Ec2).

TABLE 1
Spleen Weights and PCR Analysis of LP-BM5 for Immunized and
Non-Immunized Mice Infected or Uninfected with MAIDS at 8 Weeks

| | | | | |
|---|---|---|---|---|
| | | Spleen | | |
| | | Weight | Macroscopic | PCR Results |
| Group | Mouse No. | (mg) | Observations | LP-BM5 |

PART A - 8 weeks post infection

| | | | | |
|---|---|---|---|---|
| Control | 1 | 104 | neg | neg |
| | 2 | 108 | neg | neg |
| | 3 | 104.2 | neg | neg |
| Control virus | 7 | 395 | pos | pos |
| | 8 | 260 | pos | pos |
| | 9 | 402 | pos | pos |
| | 10 | 456 | pos | pos |
| Control | 19 | 70 | neg | neg |
| microbicide | 20 | 142 | neg | neg |
| | 21 | 122 | neg | neg |
| EV1 w/o RIBI | 25 | 396 | pos | pos |
| | 26 | 270 | pos | pos |
| | 27 | 111 | neg | neg |
| | 28 | 298 | pos | pos |
| | 29 | 540.3 | pos | pos |
| Ec1 | 35 | 110 | neg | neg |
| | 36 | 124 | neg | neg |
| | 37 | 91 | neg | neg |
| | 38 | 102 | neg | neg |
| Ev2 w RIBI | 43 | 120 | neg | neg |
| | 44 | 97 | neg | neg |
| | 45 | 106 | neg | neg |
| | 46 | 200 | pos | pos |
| | 47 | 109.2 | neg | neg |
| Ec2 | 53 | 101 | neg | neg |
| | 54 | 112 | neg | neg |
| | 55 | 110 | neg | neg |

PART B - 8 Weeks Post-Infection with Modified Cocktail

| | | | | |
|---|---|---|---|---|
| Ec2 mod | 56 | 147.2 | neg | pos |
| | 57 | 506.3 | pos | pos |
| | 58 | 123.7 | neg | neg |
| | 59 | 122.2 | neg | neg |
| | 60 | 106.6 | neg | neg |
| V mod 1 | 11 | 1050.5 | pos | pos |
| | 12 | 566.9 | pos | pos |
| | 13 | 303.3 | pos | pos |
| V mod 1 | 14 | 1301.7 | pos | pos |
| | 15 | 1360.3 | pos | pos |
| | 16 | 518.9 | pos | pos |

Groups:
“Control” means animals not immunized (medium only; no infection)
“Control virus” means animal not immunized (medium only) and infected with live LP-BM5
“Control microbicide” means animals not immunized (medium only); no infection
“Ev 1 w/o RIBI” means that the vaccination cocktail was given without the adjuvant RIBI
“Ec1” means immunized with MIC-LP-BM5 (no RIBI), no infection
“Ev 2 w RIBI” means that the vaccination cocktail was given with the adjuvant RIBI
“Ec2” means immunized with MIC-LP-BM5 (plus RIBI), no infection
“Ec 2 mod” means mice vaccinated with LP-BM5 + Microbicide + RIBI, then infected at 8 weeks post vaccination and left for 8 weeks
“V mod 1” means virus infected group, infected for 8 weeks and given vaccination cocktail LP-BM5 + Microbicide + RIBI
“V mod 2” means virus infected group, infected for 8 weeks and given mock vaccination cocktail Sc-1 + Microbicide + RIBI

Table 1 data illustrates that mice receiving the vaccine plus the adjuvant Ribi and subsequently infected with the MAIDS virus, showed an ability to resist infection as demonstrated by the lack of spleen weight increase. The question of whether spleen weight is a reliable indicator of viral infection is addressed by RT-PCR data included in Table 1. To answer this question, mouse spleen weight was compared with the presence of viral RNA in the spleen, as determined using RT-PCR.

As the data show, with the exception of three mice, there was a very strong correlation between the increase in spleen weight and the presence of viral RNA (or viral load). This correlation is primarily qualitative. Since the RT-PCR assay is not quantitative in nature, a quantitative correlation between spleen weight and viral load has not been established. The excellent qualitative correlation validates the use of spleen weight as a reliable index of infection in this model. RT-PCR amplification of the actin RNA was employed in each assay as a control. Actin is considered a stable “housekeeping” gene and is routinely used to test the integrity of the extracted cellular RNA. RNA was isolated from spleen cells and amplified by RT-PCR. In all cases a strong actin amplification was observed (not shown), confirming that the RT-PCR assays accurately reflected the LP-BM5 RNA composition in the spleen samples.

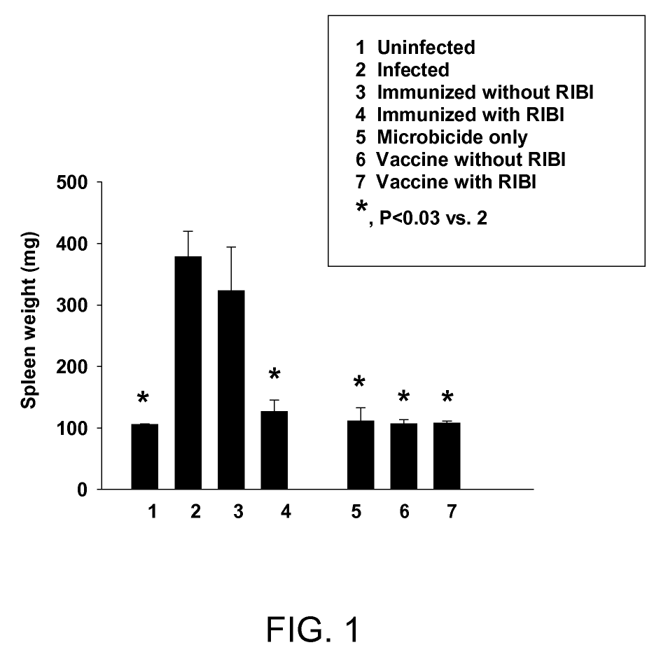

FIG. 1 illustrates spleen weight data provided in Table 1, PART A.

Table 2 illustrates spleen weight data in animals either not immunized or immunized with inactivated LP-BM5 virus (MIC-LP-BM5) prepared as described above, either with or without Ribi adjuvant. Animals were then either uninfected or infected with live LP-BM5. Spleen weight was determined at 16 weeks after infection.

Table 2 also illustrates the results of RT-PCR analysis of LP-BM5 for immunized and non-immunized mice infected or uninfected with MAIDS (“PCR Results LP-BM5” column). RNA extraction from spleen was conducted with 20-30 mg of spleen tissue, and from lymph node tissue. Spleen and lymph node tissues gave the same result.

TABLE 2
Spleen Weights and PCR Analysis of LP-BM5 for Immunized and Non-
Immunized Mice Infected or Uninfected with MAIDS at 16 Weeks

| | | Spleen Weight | Macroscopic | |
|---|---|---|---|---|
| Group | | (mg) | Observations | LP-BM5 |
| Control | 4 | 99.4 | neg | neg |
| | 5 | 126.9 | neg | neg |
| Control | 22 | 78.4 | neg | neg |
| microbicide | 23 | 82.8 | neg | neg |
| Ev 1 w/o RIBI | 30 | 1399.7 | pos | pos |
| | 31 | 87.6 | neg | neg |
| | 32 | 749.6 | pos | pos |
| | 33 | 106.2 | neg | neg |
| | 34 | 2224.6 | pos | pos |
| EC 1 | 39 | 98.2 | neg | neg |
| | 40 | 105.5 | neg | neg |
| | 41 | 94.2 | neg | neg |
| | 42 | 91.6 | neg | neg |
| EV 2 w RIBI | 48 | 103.3 | neg | neg |
| | 49 | 92.2 | neg | neg |
| | 50 | 92.0 | neg | neg |
| | 51 | 109.8 | neg | neg |
| | 52 | 160.9 | pos | pos |

Groups:
“Control” means animals not immunized (medium only), no infection
“Control microbicide” means animals not immunized (medium only), no infection
“Ev 1 w/o RIBI” means that the vaccination cocktail was given without the adjuvant RIBI
“Ec1” means immunized with MIC-LP-BM5 (no RIBI), no infection
“Ev 2 w RIBI” means that the vaccination cocktail was given with the adjuvant RIBI

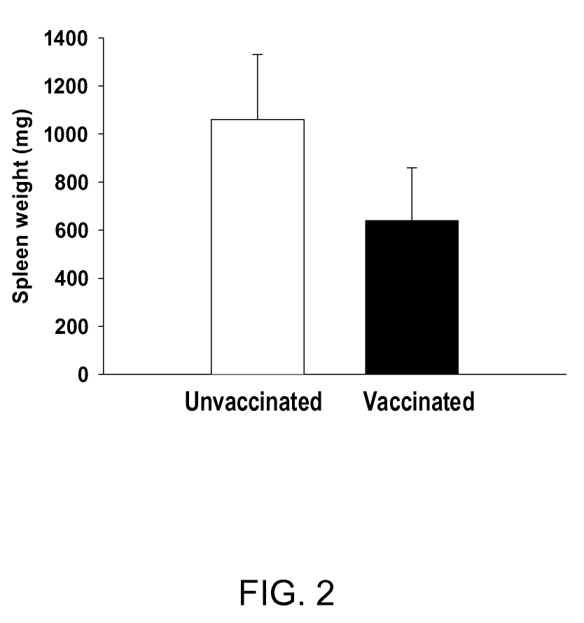

FIG. 2 illustrates spleen weight data of unvaccinated versus vaccinated animals provided in Table 1, PART B. The reduced spleen weight for vaccinated animals is evident.

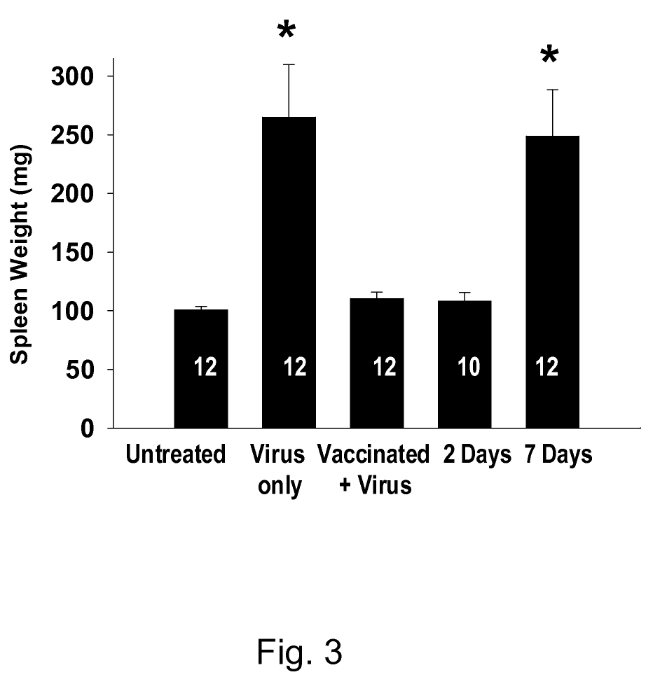

FIG. 3 illustrates spleen weight data of unvaccinated versus vaccinated animals in both prophylactic and therapeutic models. In the prophylactic model, the spleen weight of vaccinated mice challenged with live LP-BM5 (“Vaccinated+Virus”) was significantly lower than in non-vaccinated mice (“Virus only”) (110±5.5 vs. 265±45 mg, respectively, P<0.05), and was not statistically different from control (“Untreated”) (101±3 mg). Viral load determinations using RT-PCR qualitatively confirmed these observations (data not shown). In the therapeutic model, inoculation of infected mice with vaccine at 2 days post infection (“2 days”) completely blocked spleen weight increase (108±7.4 mg, P<0.05). However, delaying the first vaccine bolus to seven days post-infection (“7 days”) provided greatly diminished protection (249±40 mg, n=12). Viral load data supported these findings (data not shown).

### EXAMPLE 2

### Disruption of Zinc Finger Proteins with Various Chloramines

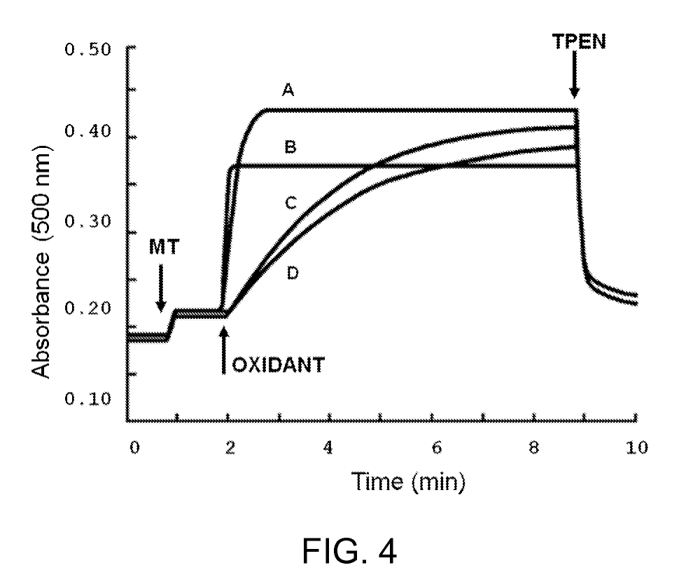

FIGS. 4 to 6 provide data illustrative of the effect of chloramines other than taurine chloramine, on disrupting zinc finger proteins. These data were obtained using a zinc-finger disruption assay, described in detail in the following publications: Fliss, H.; Menard, M., “Hypochlorous acid-induced mobilization of zinc from metalloproteins” Arch. Biochem. Biophys. 287:175-179, 1991; and Fliss, H.; Ménard, M., “Oxidant-induced mobilization of zinc from metallothionein” Arch. Biochem. Biophys. 293:195-199, 1991. Briefly, the assay is conducted as follows. The assay is performed in a cuvet containing the zinc finger protein metallothionein (MT), and the metallochromic indicator 4-(2-pyridylazo)resorcinol (PAR). When the chloramine is added to the cuvet, the zinc fingers of MT are disrupted, causing the ejection of zinc from the zinc finger. The reaction of the released zinc with PAR results in an immediate increase in PAR absorbance at 500 nm.

FIGS. 4 to 6 show an increase in PAR absorbance as soon as the chloramine is added. A chloramine forms when the natural neutrophil oxidant hypochlorous (HOCl) reacts with any available amine group (—NH₂). Thus, the data support a clear effect of several types of chloramine on zinc finger disruption.

FIG. 4 demonstrates the relative activity of the chloramine of ammonia (monochloramine) “A”; the oxidant hypochlorous acid (HOCl) “B”; taurine chloramine “C”; and the chloramine of the amino acid alanine “D”, on zinc-finger disruption, each of which was present at a concentration of 25 μmol/L. Each of these chloramines shows an increase in PAR absorbance attributable to the chloramine functionality.

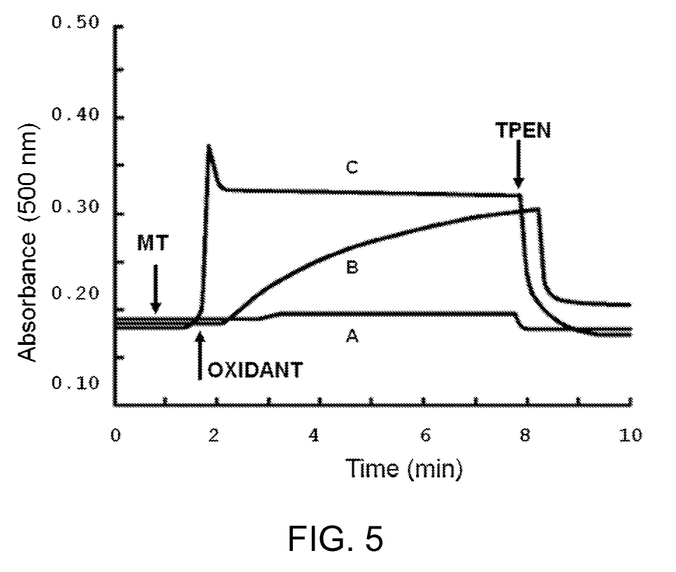

FIG. 5 illustrates the activity of “A” the amino acid phenylalanine at 500 μmol/L when compared to “B” phenylalanine chloramine at 100 μmol/L; and “C” HOCl at 100 μmol/L on the release of zinc in the zinc-finger assay. MT is shown at 10 μL of 1 mg/mL prior to addition of A, B or C. TPEN (a zinc chelator) is shown at 10 μL of a 2 mmol/L solution (20 μmol/L final concentration). The effect of phenylalanine alone is negligible, as compared to the baseline absorbance, whereas the phenylalanine chloramine shows a significant and increasing effect over time. The effect of hypochlorous acid alone is rapid, consistent with the data shown in FIG. 3.

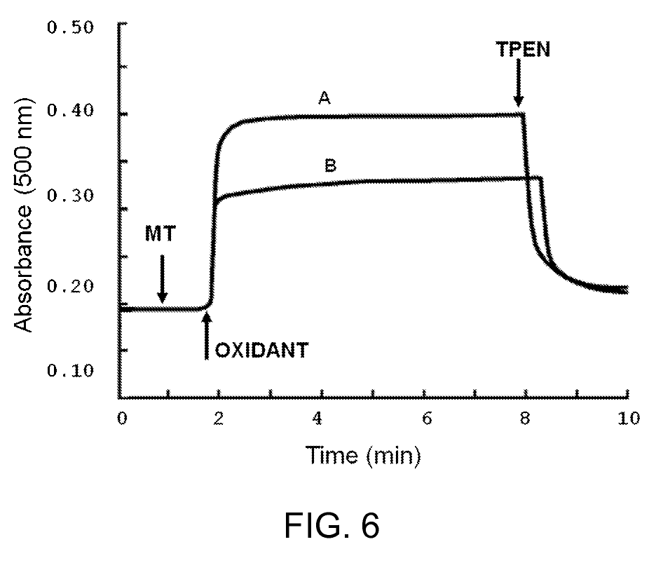

FIG. 6 shows a comparison between PAR absorbance due to zinc release for hypochlorous acid (HOCl) at 25 μmol/L and the chloramine of adenosine (Aden) at 25 μmol/L. Adenosine was chosen for this comparison because it is a common nucleoside. These data illustrate the rapid effect of adenosine chloramine, and by extension the chloramine derivatives of other nucleosides, in mobilization of zinc from zinc-finger proteins, and thus illustrates utility for nucleoside chloramines in inactivating a live retrovirus containing zinc fingers therein.

### EXAMPLE 3

### Killing of HIV by Taurine Chloramine in Human Blood

We have shown that taurine chloramine at the non-injurious concentrations of 2 mM or 5 mM can totally inactivate very high titers of human immunodeficiency virus (HIV) in human blood under conditions that simulate the preparation of autologous therapeutic vaccines. These data confirm that, in addition to LP-BM5, the murine immunodeficiency virus, other retroviruses are similarly susceptible to chloramine inactivation. The following are the details of the experiment.

### Methods

Commercially available HIV-1 (strain IIIB, Advanced Biotechnologies Inc. MD) was added to fresh human red blood cell (RBC) suspensions (70% hematocrit) to give a final concentration of 1×10⁶ TCID₅₀ units/ml, and was then treated for 1 h at room temperature with either 2 or 5 mM of taurine chloramine. To determine the infectious properties of the treated HIV, the RBC suspensions were then centrifuged (100×g, 10 min) and aliquots of the supernatant, containing the chloramine-treated HIV, were withdrawn and added to phytohemagglutinin (PHA)-activated human peripheral blood mononuclear cells (PBMC). The HIV-treated PBMC cultures were maintained for a period of 21 days. Aliquots of the culture medium were removed at 0 min, and 7, 14, and 21 days for analysis of HIV production using an ELISA assay for p24, an HIV protein (Coulter, Miami, Fla.). Control samples were treated identically but with the omission of taurine chloramine.

### Results

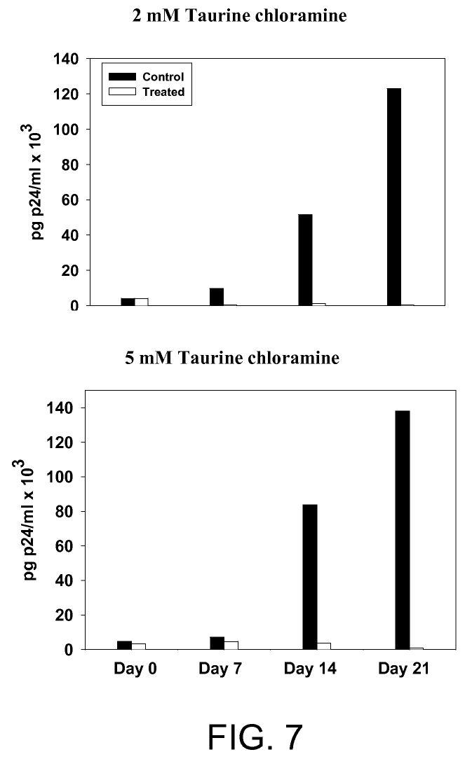

FIG. 7 provides data illustrative of the effect of taurine chloramine on the infectious properties of HIV in human blood. Either 2 mM or 5 mM taurine chloramine were able to totally eliminate HIV infectivity as illustrated by the lack of p24 production in the infected PBMC cultures for up to 21 days, indicating a greater than 6 log reduction in HIV production. In contrast, control samples treated in identical fashion, but with the omission of taurine chloramine, showed exponential growth of HIV in the infected PBMC. Each panel represents the data from one test culture and one control culture. Both the 2 mM and 5 mM runs were repeated and gave results identical to the ones shown.

The above-described embodiments of the present invention are intended to be examples only. Alterations, modifications and variations may be effected to the particular embodiments by those of skill in the art without departing from the scope of the invention, which is defined solely by the claims appended hereto.

### REFERENCES

U.S. Pat. No. 6,706,729 (Turpin et al.) “Thiolesters and uses thereof”. Issued Mar. 14, 2004.
Arthur, L. O. et al. “Chemical Inactivation of Retroviral Infectivity by Targeting Nucleocapsid Protein Zinc Fingers: A Candidate SIV Vaccine” Aids Research and Human Retroviruses 14(S3): S311-S319, 1998.
Fliss, H.; Ménard, M., “Hypochlorous acid-induced mobilization of zinc from metalloproteins” Arch. Biochem. Biophys. 287:175-179, 1991.
Fliss, H.; Ménard, M., “Oxidant-induced mobilization of zinc from metallothionein” Arch. Biochem. Biophys. 293:195-199, 1991.
Hulier E, et al. “Quantitative assessment of murine retrovirus LP-BM5 infection in MAIDS by PCR and anion exchange HPLC” Journal of Virological Methods. 60:109-117,
Matteucci, D. et al. “Vaccination protects against in vivo-grown feline immunodeficiency virus even in the absence of detectable neutralizing antibodies” Journal of Virology 70(1):617-622, 1996.
Nagl, M., Teuchner, B., Pottinger, E., Ulmer, H., and Gottardi, W. “Tolerance of N-chlorotaurine, a new antimicrobial agent, in infectious conjunctivitis—A phase II pilot study”. Opthalmologica 2000, 214:111-114.
Nagl, M., Hess, M. W., Pfaller, K., Hengster, P., and Gottardi, W. “Bactericidal activity of micromolar-chlorotaurine: Evidence for its antimicrobial function in the human defense system” Antimicrob. Agents Chemother. 2000, 44:2507-2513.
Robbins, G. K., et al. “Augmentation of HIV-1-specific T helper cell responses in chronic HIV-1 infection by therapeutic immunization” AIDS 1993, 17:1121-1126.
Rossio, J. L. et al. “Inactivation of Human Immunodeficiency Virus Type 1 Infectivity with Preservation of Conformational and Functional Integrity of Virion Proteins” Journal of Virology 72(10):7992-8110, 1998.
Teuchner et al. “Tolerance and efficacy of the new antimicrobial agent N-chlorotaurine in viral keratoconjunctivitis” Annual Meeting of the Association for Research in Vision and Opthalmology, (abstract) Mar. 15, 2001 page S578 (XP001027296).
U.S. Pat. No. 6,001,555 (Henderson et al.) “Method for identifying and using compounds that inactivate HIV-1 and other retroviruses by attacking highly conserved zinc fingers in the viral nucleocapsid protein”. Issued Dec. 14, 1999.
International Patent Application No. PCT/CA01/01421 (Fliss) “Chloramines as antiviral agents”. Published Apr. 11, 2002.
International Patent Application No. PCT/EP96/01356 (Bendinelli et al.) “Vaccine for the immunoprophylaxis of the infection from feline immunodeficiency virus in the domestic cat”. Published Oct. 3, 1996.
German Patent application DE 197 12 565 A1 (Steif).
German Patent application DE 198 16 102 C1 (Gottardi).

What is claimed is:

1. A viral vaccine comprising a cell lysate or culture medium containing a whole-killed virus suspension and a pharmaceutically acceptable carrier, said virus having been killed by exposure to a chloramine compound at a level adequate to inactivate zinc finger proteins.

2. The viral vaccine according to claim 1 wherein said virus is a retrovirus

3. The viral vaccine according to claim 1 wherein said vaccine additionally comprises a chloramine compound.

4. The viral vaccine according to claim 1 wherein said chloramine compound is selected from the group consisting of taurine chloramine, adenosine chloramine, phenylalanine chloramine, and alanine chloramine.

5. The viral vaccine according to claim 1 which is a prophylactic vaccine.

6. The viral vaccine according to claim 1 which is a therapeutic vaccine.

7. The viral vaccine according to claim 1 which is an autologous vaccine.

8. A method for forming a viral vaccine comprising the steps of:
obtaining a sample of a virus; and
exposing said virus sample to a chloramine compound in a quantity adequate to inactivate zinc finger proteins of said virus.

9. The method according to claim 8 wherein said chloramine compound is selected from the group consisting of taurine chloramine, adenosine chloramine, phenylalanine chloramine, and alanine chloramine.

10. The method according to claim 8 wherein said virus sample is exposed to a chloramines at a concentration of about 1 to 10 mmol/L.

11. The method according to claim 8, wherein after the step of exposing the virus sample to a chloramine compound, a whole-killed virus is obtained.

12. A viral vaccine formed according to the method of claim 8 wherein said virus is a retrovirus.

13. The viral vaccine according to claim 12 wherein said retrovirus is HIV

14. A viral vaccine comprising a whole-killed virus formed according to the method of claim 12 wherein said virus is a retrovirus.

15. A method of autologous vaccination of a subject against a virus, said method comprising the steps of:
obtaining a sample of a virus from a subject;
exposing said virus sample to a chloramine compound in a quantity adequate to inactivate zinc finger proteins of said virus to form a vaccine; and
providing said vaccine to the subject.

16. The method according to claim 15, wherein the step of obtaining a sample of a virus additionally comprises amplifying said virus in cell culture and preparing extracts of said cell culture to be used in the step of exposing.

17. The method of claim 16, wherein said cell culture comprises autologous or heterologous cell culture.

18. The method of autologous vaccination according to claim 15, wherein said chloramine compound is selected from the group consisting of taurine chloramine, adenosine chloramine, phenylalanine chloramine, and alanine chloramine.

19. The method of claim 15 wherein said virus is a retrovirus.

20. The method of claim 19 wherein said retrovirus is HIV.


SEQUENCE LISTING (2 sequences)

SEQ ID NO: 1 - DNA, 20 bp, Artificial
  forward primer (upstream or sense)
cctttatcgacacttccctt

SEQ ID NO: 2 - DNA, 20 bp, Artificial
  reverse primer (downstream)
ccgcctcttcttaactggtc